(19) United States
(12) Patent Application Publication
(10) Pub. No.: US 2019/0127693 A1
(43) Pub. Date: May 2, 2019

(54) METHOD OF MANUFACTURING CELL-NANOSCALE THIN FILM COMPOSITE

(71) Applicant: TOHOKU UNIVERSITY, Sendai-shi, Miyagi (JP)

(72) Inventors: Hirokazu KAJI, Miyagi (JP); Jin SUZUKI, Miyagi (JP); Nobuhiro NAGAI, Miyagi (JP); Toshiaki ABE, Miyagi (JP)

(21) Appl. No.: 16/094,777

(22) PCT Filed: Apr. 21, 2017
(86) PCT No.: PCT/JP2017/016010

§ 371 Date: Oct. 18, 2018

(30) Foreign Application Priority Data
Apr. 22, 2016 (JP) 2016-086455

(51) Int. Cl. C12N 5/079; C12M 3/00; C12M 1/26; A61L 27/36

(52) U.S. Cl. CPC C12N 5/0621; C12M 21/08; C12N 2539/10; A61L 27/3691; C12N 2533/30; C12M 33/00

(57) ABSTRACT

Provided is a novel method of manufacturing a cell-nanoscale thin film composite in which the cell-nanoscale thin film composite can be peeled from a substrate at a controlled timing. The method of manufacturing a cell-nanoscale thin film composite comprises culturing a cell in a cell culture base material in which a nanoscale thin film is provided on an electrode substrate with a self-assembled monolayer interposed therebetween, and reductively desorbing the self-assembled monolayer from the electrode substrate by applying an electric potential to the electrode substrate at a desired timing, so that the cell-nanoscale thin film composite is released.

### TECHNICAL FIELD

The present invention relates to means for peeling a cell-nanoscale thin film composite from a substrate at a controlled timing, and a method of manufacturing a cell-nanoscale thin film composite using the means.

### BACKGROUND ART

In recent years, development of cell transplantation therapy for intractable disease has been extensively promoted in the field of regenerative medicine. For example, an attempt has been made to treat various organs and tissues by inducing needed cells from iPS cells to prepare a cell sheet, and transplanting the cell sheet in an affected part. Heretofore, clinical studies and clinical trials for corneal disease, esophageal disease, heart disease, periodontal disease, cartilage disease, and the like using cell sheets have been conducted, and expansion of the application range to lung disease, ear disease, lever disease, pancreas disease, and the like in addition to the above-mentioned diseases is being considered.

In preparation and use of a cell sheet, it is required to peel the prepared cell sheet from a substrate in an intact state. Currently, as a method of recovering a cell sheet, studies are promoted, for example, on use of a temperature-responsive cell culture plate (Non Patent Literature 1) and on a method in which a cell sheet is electrochemically peeled from a substrate using a so-called self-assembled monolayer (SAM) (Non Patent Literature 2 and Patent Literatures 1 and 2).

A polymer nanoscale thin film (hereinafter, referred to as a “nanoscale thin film”) belongs to a relatively new category of soft nanoscale materials which are studied in the field of polymer physics (Non Patent Literature 3). The nanoscale thin film is a thin film having a thickness of several tens to several hundreds nm, and has high flexibility. In addition, since the nanoscale thin film is thin and flexible, it can follow an irregular surface, and adhere to a variety of surfaces by means of a large intermolecular force. The present inventors have already found and reported that when a nanoscale thin film is prepared using a polylactic acid-glycolic acid copolymer (PLGA) having favorable biocompatibility and biodegradability, and cells are cultured on the nanoscale thin film, a cell-nanoscale thin film composite excellent in flexibility, extensibility, adhesiveness and biocompatibility can be obtained (Patent Literature 3). The cell-nanoscale thin film composite makes it possible to effectively deliver cells by overcoming the disadvantage of a cell sheet: the cell sheet is fragile, and easily broken (Non Patent Literature 4).

On the other hand, in order to prepare the cell-nanoscale thin film composite, it is required to culture cells on the nanoscale thin film for a long period of time. When cells having scaffold properties are cultured, it is necessary that the nanoscale thin film adhere to a substrate during a culture period, but in a situation where the cell-nanoscale thin film composite is used (at treatment and operation sites), it is desired to peel the cell-nanoscale thin film composite from the substrate at any timing. Therefore, a technique capable of controlling peeling of a cell-nanoscale thin film composite from a substrate is desired.

### CITATION LIST

### Patent Literature

Patent Literature 1: JP Patent Publication (Kokai) No. 2008-295382

Patent Literature 2: WO 2012/033181

Patent Literature 3: WO 2014/208778

### Non Patent Literature

Non Patent Literature 1: Hideaki Sakai et al., “Regenerative Medicine by the World's First “Cell Sheet Engineering” Technology Developed in Japan,” Tokugikon, No. 271

Non Patent Literature 2: Inaba R. et al., Biomaterials, 30, 21, 3573-9, 2009

Non Patent Literature 3: J. A. Forrest et al., Advances in Colloid and Interface Science, 94, 1-3, 167-195, 2001

Non Patent Literature 4: T. Fujie et al., Adv Mater, 26, 1699-1705, 2014

### SUMMARY OF INVENTION

### Technical Problem

An object of the present invention is to provide a novel means capable of controlling a timing at which a cell-nanoscale thin film composite is peeled from a substrate.

### Solution to Problem

The present inventors have extensively conducted studies for solving the above-described problems, and resultantly found that when a cell-nanoscale thin film composite is provided on an electrode substrate as a substrate with a self-assembled monolayer (SAM) interposed therebetween, the cell-nanoscale thin film composite can be released from the substrate by electrochemically peeling the SAM from the electrode substrate, and recovered, leading to completion of the present invention.

Specifically, the present invention includes the following inventions.
[1] A method of manufacturing a cell-nanoscale thin film composite, the method comprising the steps of:

culturing a cell on a nanoscale thin film in a cell culture base material in which the nanoscale thin film is provided on an electrode substrate with a self-assembled monolayer interposed therebetween;

peeling the self-assembled monolayer from the electrode substrate by applying an electric potential to the electrode substrate so that the self-assembled monolayer is reductively desorbed from the electrode substrate; and

recovering a cell-nanoscale thin film composite released from the electrode substrate as the self-assembled monolayer is peeled.
[2] The method according to [1], wherein the nanoscale thin film comprises a biocompatible polymer.
[3] The method according to [2], wherein the biocompatible polymer is a polylactic acid-glycolic acid copolymer.
[4] The method according to any one of [1] to [3], wherein the self-assembled monolayer comprises an alkane thiol or a derivative thereof, or cysteine.
[5] The method according to any one of [1] to [4], wherein the electrode substrate is a porous material.
[6] The method according to [5], wherein the porous material that is the electrode substrate is a porous film.
[7] A cell culture base material in which a nanoscale thin film is provided on an electrode substrate with a self-assembled monolayer interposed therebetween.
[8] The cell culture base material according to [7], wherein the electrode substrate is a porous material.
[9] The cell culture base material according to [8], wherein the porous material that is the electrode substrate is a porous film.
[10] A cell culture apparatus comprising a cell culture base material in which a nanoscale thin film is provided on an electrode substrate with a self-assembled monolayer interposed therebetween, and a counter electrode and a power source which are used for applying an electric potential to the electrode substrate.
[11] The cell culture apparatus according to [10], wherein the electrode substrate is a porous material.
[12] The cell culture apparatus according to [11], wherein the porous material that is the electrode substrate is a porous film.
[13] A cell-nanoscale thin film composite in which a self-assembled monolayer, a nanoscale thin film and a cell are stacked in this order.

### Advantageous Effects of Invention

According to the present invention, a cell-nanoscale thin film composite can be released from a substrate at any timing, and recovered.

### BRIEF DESCRIPTION OF DRAWINGS

FIG. 1 shows a simplified diagram of a method of preparing a nanoscale thin film.

FIG. 2 shows a simplified diagram of a method of preparing an electrode substrate having a SAM.

FIG. 3 shows cyclic voltammograms obtained by applying an electric potential to an electrode substrate having each SAM, in which (A) illustrates a first sweep and (B) illustrates a second sweep.

FIG. 4 shows a photographic view of a cell culture base material in which a nanoscale thin film is provided on an electrode substrate having a SAM (scale bar: 5.0 mm).

FIG. 5 is a photographic view illustrating a state in which a nanoscale thin film is peeled from an electrode substrate by applying an electric potential, in which (a) illustrates a state before the electric potential is applied;(b) illustrates just when the nanoscale thin film is peeled; and (c) illustrates a state after the nanoscale thin film is peeled.

FIG. 6 is a photographic view illustrating a state in which a cell-nanoscale thin film composite is peeled from an electrode substrate by applying an electric potential, in which (a) illustrates a state before the electric potential is applied; (b) illustrates just when the cell-nanoscale thin film composite is peeled; and (c) illustrates a state after the cell-nanoscale thin film composite is peeled.

FIG. 7 is a graph illustrating a cell survival rate before and after a cell-nanoscale thin film composite is desorbed from an electrode substrate.

FIG. 8 illustrates a gold porous electrode in which (a) is a photograph and (b) is a SEM image.

FIG. 9 is a photograph illustrating a state in which a nanoscale thin film is desorbed from a porous electrode.

FIG. 10 is a graph illustrating a time required for desorption of a nanoscale thin film from an electrode substrate.

FIG. 11 is a diagram illustrating a method of subretinally transplanting a cell-nanoscale thin film composite by a syringe needle.

FIG. 12 is an optical coherence tomography (OCT) image of the retina after delivery of a cell-nanoscale thin film composite.

FIG. 13 is a photograph of a circular nanoscale thin film of a posterior eye segment, which is observed in an extracted eyeball.

FIG. 14 shows an image of a frozen section of the retina of an extracted eyeball.

FIG. 15 is an image of a retina section stained with hematoxylin eosin.

### DESCRIPTION OF EMBODIMENTS

1. Cell Culture Base Material

The “cell culture base material” in the present invention has a structure in which a nanoscale thin film is provided on an electrode substrate as a substrate with a self-assembled monolayer interposed therebetween, and cells can adhere onto the nanoscale thin film, and the cell culture base material can be used as a material serving as a scaffold in cell culture.

The “nanoscale thin film” in the present invention means a sheet comprising biocompatible polymer, the sheet having a thickness of less than 500 nm, preferably about 400 nm or less, more preferably about 300 nm or less, still more preferably about 200 nm or less. The lower limit of the thickness of the “nanoscale thin film” is not particularly limited, and may be about 20 nm or more, preferably about 40 nm or more, more preferably about 60 nm or more, still more preferably about 80 nm or more, even more preferably about 100 nm or more. For example, the “nanoscale thin film” in the present invention may be a sheet comprising a biocompatible polymer, the sheet having a thickness of about 20 nm to 300 nm, preferably about 100 nm to 200 nm.

Examples of the “biocompatible polymer” usable in the present invention include polylactic acid, polyglycolic acid, polyhydroxy butyric acid, polycaprolactone, polybutylene succinate, polydioxanone, polydimethylsiloxane, polymethyl methacrylate, polystyrene, polyvinyl acetate, poly(3,4-ethylenedioxythiophene), proteins (collagen, gelatin, laminin, fibronectin and elastin), polysaccharides (chitosan, alginic acid, hyaluronic acid, chondroitin sulfate and cellulose), nucleic acids (DNA and RNA), and copolymers thereof. The biocompatible polymer is preferably a biodegradable polymer, especially preferably a polylactic acid-glycolic acid copolymer (hereinafter, referred to as “PLGA”).

The shape of the nanoscale thin film in the present invention is not particularly limited, and may be any shape such as a circle, an ellipse, a polygon (e.g., rectangle, square, pentagon, or hexagon) or a combination thereof. For example, the diameter or the length of the longest diagonal of the nanoscale thin film may be about 10 mm to 20 mm, preferably about 50 mm to 15 mm, more preferably about 100 mm to 10 mm, still more preferably about 150 mm to 5 mm, even more preferably about 200 mm to 3 mm, especially preferably about 300 mm to 1 mm (but is not limited thereto). When the nanoscale thin film has such a shape (size), the cell-nanoscale thin film composite as a final product can be suctioned and discharged through a capillary, so that the transplantation operation of the composite in the living body, or the like can be facilitated.

A surface of the nanoscale thin film, which carries cells, can be coated with an extracellular matrix that promotes adhesion and proliferation of cells. Examples of the “extracellular matrix” usable in the present invention include I-type collagen, IV-type collagen, fibronectin, poly-D-lysine (PDL), laminin and poly-L-ornithine/laminin (PLO/LM).

A functional substance can be provided in the nanoscale thin film or on a surface thereof. The “functional substance” means a substance having a function of controlling proliferation, differentiation, bioactivity, and the like of cells (e.g., a protein, a polypeptide, or a compound), or a substance enabling visualization of the nanoscale thin film. Examples of the functional substance include growth/proliferation factors (e.g., fibroblast growth factor (FGF), epidermal growth factor (EGF), bone morphogenetic protein (BMP), nerve growth factor (NGF), and brain-derived neurotrophic factor (BDNF)), ocular hypotensive agents, neuroprotective agents, antibiotics, anticancer agents and visualization probes (contrast media, nanoparticles, fluorescent dyes, and the like).

In addition, nanoparticles composed of a metal, a semiconductor, a ceramic, a magnetic material, or the like, preferably magnetic material nanoparticles can be provided in the nanoscale thin film or on a surface thereof. The nanoparticles have a particle size of about 1 nm to 500 nm, preferably about 1 nm to 50 nm. When the nanoscale thin film has such nanoparticles, irregularities stemming from the nanoparticles can be formed on a surface of the nanoscale thin film. By forming irregularities on the surface of the nanoscale thin film, the cell-adherable surface area can be increased, and the proliferation activity of cells can be enhanced. In addition, when magnetic material nanoparticles are included in the nanoscale thin film, the nanoscale thin film can be moved and collected by means of a magnetic force, so that operability of the cell-nanoscale thin film composite as a final product can be improved.

The “electrode substrate” usable in the present invention may be one allowing a self-assembled monolayer to be bonded thereto via a thiolate, and having electrical conductivity so that the electrode substrate functions as a working electrode, and for example, one or more materials (electrode materials) selected from noble metals such as gold, silver, platinum and copper, semiconductors such as silicon, silicon carbide and zinc selenide, and metal oxides such as tin oxide, zinc oxide and indium oxide can be used. The electrode substrate may be composed of an electrode material, or formed by covering part or the whole of a surface of another material (support substrate) with an electrode material. The other material is not particularly limited, and a synthetic resin, a metal or an inorganic material (glass, silicon, ceramic, or the like) can be used. The other material may have electrical conductivity, or have no electrical conductivity.

The shape and size of the electrode substrate are not particularly limited as long as the nanoscale thin film can be supported, and the electrode substrate may have any shape, and have a shape and/or size identical to or different from the shape and/or size of the nanoscale thin film.

In addition, the electrode substrate may have a shape of a porous material, mesh, or the like through which a culture medium can pass. When the electrode substrate has a shape of a porous material, mesh, or the like, the culture medium can enter between the electrode substrate and the self-assembled monolayer not only from an edge part but also by passing through the electrode substrate in application of an electric potential to the electrode substrate, so that an electrolyte solution can be quickly supplied to an electrode surface, and peeling of the self-assembled monolayer from the electrode substrate can be facilitated. In addition, for example, a porous film (porous electrode substrate) can be used as the porous material, and examples of the porous film include a porous thin film coated with gold. The pore size of the porous film is several hundreds nm to several μm, preferably about 1 μm.

The “self-assembled monolayer” in the present invention is a highly oriented nanoscale monolayer formed such that a plurality of hydrophobic compounds bonded to the electrode substrate and/or nanoscale thin film are integrated at a high density by intermolecular interaction. Such a self-assembled monolayer may be generally referred to as “SAM” (abbreviation of self-assembled monolayer), and herein, the self-assembled monolayer may be referred to simply as “SAM.” Herein, a compound to be used for forming a SAM may be referred to as a SAM compound.

Examples of the SAM compound include linear hydrophobic molecules which have a thiol group (—SH group) at the end, or have a sulfide (—S—) or disulfide (—S—S—) structure, and can be bonded to the electrode substrate via such a group or structure. Preferably, an alkane thiol having 4 to 20 carbon atoms, e.g., about 4 to 15 carbon atoms, or cysteine can be used as the SAM compound. Cysteine is especially preferable. When the SAM comprises cysteine, the SAM can be peeled from a surface of the electrode substrate by application of a relatively low reduction electric potential.

The SAM compound may be a derivative modified with a functional group which can be bonded to the nanoscale thin film. A functional group to be used can be appropriately selected according to a nanoscale thin film to be used, and an amino group, a carboxyl group, a hydroxyl group, an aldehyde group, or the like can be used. For example, when the nanoscale thin film comprises PLGA, the SAM compound can be modified with a carboxyl group so that the SAM compound can be bonded to the nanoscale thin film.

The SAM comprising the SAM compound can be bonded to both the electrode substrate and the nanoscale thin film. Accordingly, the nanoscale thin film is bonded to the SAM, and adsorbed to and provided on the electrode substrate bonded to the SAM, with the SAM interposed therebetween.

The cell culture base material according to the present invention can be obtained by a method including the following steps. Hereinafter, for the sake of convenience, a step of preparing a nanoscale thin film is designated as [1], and a step of preparing an electrode substrate is designated as [2], but the order of these steps is not particularly limited, and the step of preparing an electrode substrate may be carried out before the step of preparing a nanoscale thin film, or both the steps may be carried out in parallel.

[1] Step of Preparing Nanoscale Thin Film

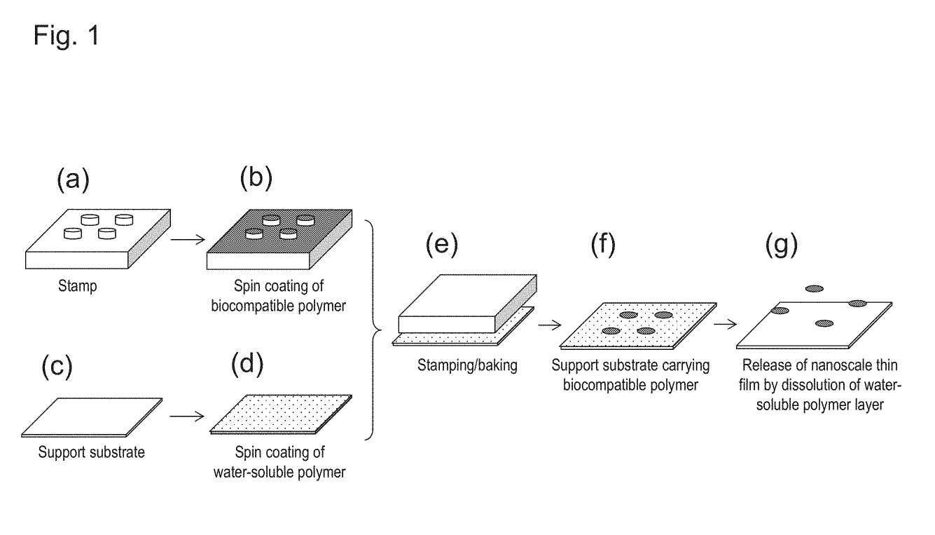

The nanoscale thin film in the present invention can be prepared on the basis of a known method (WO 2014/208778), and a preparation method using a microstamp method and a spin coating method in combination is shown below and in FIG. 1, but the method of preparing a nanoscale thin film is not limited to such a method.

(i) A substrate convexly inscribed with a predetermined pattern (hereinafter, referred to as a “stamp”) is prepared using, for example, polydimethylsiloxane (PDMS), a metal, silicon, or glass. The stamp can be prepared using a photolithography technique in accordance with a conventional method. For example, a substrate surface is coated with long-chain hydrophobic molecules of octadecyltrimethoxysilane (ODMS), octadecyldimethylchlorosilane, trialkoxyhexadecylsilane, and the like, followed by applying a positive photoresist thereon. Next, the resist is exposed (electron irradiation, ultraviolet irradiation, X-ray irradiation, or the like) through a photomask. Subsequently, the resist on the base is developed, and the resist on a photosensitized region is removed. Long-chain hydrophobic molecules on a region which is not protected with the resist are removed by O₂ plasma treatment, CO plasma treatment, or reactive ion etching treatment using a halogen gas. Finally, the resist is removed using acetone, tetrahydrofuran (THF), dichloromethane, or the like, whereby a stamp can be obtained (shown as (a) in FIG. 1).

(ii) A biocompatible polymer layer is formed on a surface of the obtained stamp (a stamp surface inscribed with the pattern). A biocompatible polymer or a constituent element of the biocompatible polymer (e.g., a monomer as a constituent element of the biodegradable polymer) (hereinafter, referred to as a “biocompatible polymer etc.”) is dissolved in an appropriate solvent (e.g., dichloromethane, chloroform, acetone, or ethyl acetate) at a concentration of 1 mg/mL to 100 mg/mL, preferably 5 mg/mL to 40 mg/mL, and the solution of the biocompatible polymer etc. is applied to a stamp surface by a spin coating method. By adjusting the rotation speed and rotation time of a spin-coater, the thickness of the biocompatible polymer etc. applied on the stamp surface can be adjusted, so that the thickness of the resulting nanoscale thin film can be adjusted.

The applied biocompatible polymer etc. is then polymerized and/or crosslinked, whereby a layer composed of a biocompatible polymer can be formed on the stamp surface (shown as (b) in FIG. 1). Here, examples of the “polymerization” may include condensation polymerization, polyaddition, addition condensation, ring-opening polymerization, addition polymerization (radical polymerization, anionic polymerization and cationic polymerization), thermal solid phase polymerization, photopolymerization, radiation polymerization and plasma polymerization. The “crosslinking” can be performed using a known crosslinking agent (e.g., an alkyldiimidate, an acyldiazide, a diisocyanate, a bismaleimide, a triazinyl, a diazo compound, or a glutaraldehyde).

(iii) A support substrate including a water-soluble sacrificial layer is prepared. One surface of the support substrate (e.g., silicon or glass) (shown as (c) in FIG. 1) is coated with a water-soluble polymer such as polyvinyl alcohol (PVA) or a derivative thereof, polyisopropyl acrylamide or a derivative thereof, polyether or a derivative thereof, a polysaccharide, a polymer electrolyte or a salt thereof. The coating can be performed by applying the water-soluble polymer on the support substrate by a casting method, a spin coating method, or the like, and drying the water-soluble polymer. Accordingly, a support substrate including a water-soluble sacrificial layer soluble in an aqueous solvent can be obtained (shown as (d) in FIG. 1).

(iv) The biocompatible polymer layer on the stamp surface is stamped and baked on the water-soluble sacrificial layer of the support substrate (shown as (e) in FIG. 1). The baking of the biocompatible polymer on the water-soluble sacrificial layer can be performed by heat treatment. Accordingly, the biocompatible polymer can be transferred onto the water-soluble sacrificial layer while the pattern is maintained, whereby a support substrate carrying a biocompatible polymer (biocompatible polymer-carrying support substrate) can be obtained (shown as (f) in FIG. 1).

(v) The biocompatible polymer-carrying support substrate is immersed in water to dissolve the water-soluble sacrificial layer, whereby the biocompatible polymer can be released from the support substrate to obtain a nanoscale thin film having a predetermined pattern (shown as (g) in FIG. 1).

[2] Step of Preparing Electrode Substrate having SAM

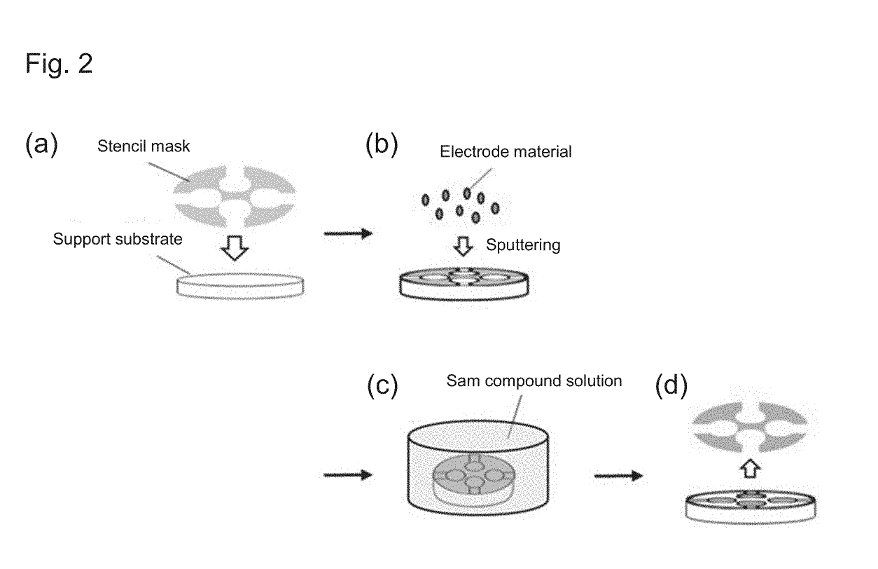

A method of preparing an electrode substrate using a sputtering method is schematically shown below and in FIG. 2, but the method of preparing an electrode substrate is not limited to such a method.

(i) A stencil mask from which a predetermined pattern is cut out is placed on a support substrate (synthetic resin, metal or inorganic material (glass, silicon, ceramic, or the like)), and on the substrate, an electrode material is deposited by sputtering to form a thin film (shown as (a) and (b) in FIG. 2). The sputtering can be performed using a known method (a bipolar sputtering method, a magnetron sputtering method, a DC sputtering method, an RF (radio frequency) sputtering method, or the like). By adjusting the sputtering time, the thickness of the electrode material deposited on the support substrate can be adjusted. To improve deposition properties of the electrode material to the support substrate, the support substrate may be coated in advance with another metal such as titanium or nickel before deposition of the electrode material.

(ii) A SAM compound is dissolved in an appropriate solvent (e.g., water, alcohol (such as ethanol), acetone, or ethyl acetate) at a concentration of 0.1 to 10 mM, preferably 1 to 2 mM, the support substrate on which the electrode material is deposited is immersed in the SAM compound solution (shown as (c) in FIG. 2), and the SAM compound is bonded to the electrode material via a thiolate to form a SAM on a surface of the electrode material.

(iii) The stencil mask is removed, whereby an electrode substrate having a predetermined pattern and having a SAM on a surface thereof can be obtained (shown as (d) in FIG. 2).

[3] Step of Obtaining Cell Culture Base Material

In water, the nanoscale thin film obtained in [1] is spread and placed on the electrode substrate obtained in [2] (more specifically on the region on which the electrode material is deposited), the nanoscale thin film is taken out from the water, and dried, and the nanoscale thin film and the SAM are adsorbed to each other. Accordingly, a cell culture base material can be obtained which has a structure in which a nanoscale thin film is adsorbed to and provided on an electrode substrate as a substrate with a SAM interposed therebetween.

Alternatively, a nanoscale thin film may be formed on an electrode substrate having a SAM in “[2] Step of preparing electrode substrate having SAM.” Specifically, a SAM is formed in step (ii) in “[2] Step of preparing electrode substrate having SAM,” and a solution of the biocompatible polymer etc. is then applied to the surface of the SAM by the same method as in step (ii) in “[1] Step of preparing nanoscale thin film.” The applied biocompatible polymer etc. is then polymerized and/or crosslinked to form a nanoscale thin film comprising a biocompatible polymer on the SAM surface. Finally, the stencil mask is removed, whereby a cell culture base material can be obtained which has a predetermined pattern, and has a structure in which a nanoscale thin film is adsorbed to and provided on an electrode substrate as a substrate with a SAM interposed therebetween.

A surface of the nanoscale thin film of the cell culture base material can be coated with an extracellular matrix which promotes adhesion and proliferation of cells. The coating of the extracellular matrix can be performed by applying a solution of the extracellular matrix in an appropriate solvent (at a concentration of, for example, 0.01 μg/mL to 5 μg/mL) on the nanoscale thin film by a spin coating method or the like, and then drying the solution.

2. Cell-Nanoscale Thin Film Composite

In the present invention, the “cell-nanoscale thin film composite” is a composite having a structure in which cells are carried on a nanoscale thin film. More specifically, the “cell-nanoscale thin film composite” in the present invention is a composite having a structure in which a self-assembled monolayer, a nanoscale thin film and cells are stacked in this order.

In the present invention, examples of cells which can be carried on the nanoscale thin film include cells which can be transplanted in patients by cell transplantation therapy (except for cells floating in body fluid), and examples of such cells include retinal pigment epithelium (RPE) cells, photoreceptor cells, liver cells, cardiac muscle cells, skeletal muscle cells, smooth muscle cells, vascular endothelial cells, renal cells, islet cells, epidermal cells and nerve cells. These cells may be cells isolated from a patient in whom the cell-nanoscale thin film composite according to the present invention is introduced/transplanted, or cells derived from ES cells, stem cells or iPS cells.

The cell-nanoscale thin film composite according to the present invention can be obtained by culturing cells using the cell culture base material, and then peeling the SAM, to which the nanoscale thin film carrying cells are bonded, from the electrode substrate.

The peeling of the SAM from the electrode substrate can be performed by applying an electric potential so that the SAM is reductively desorbed.

The electric potential to be applied to the electrode substrate may be an electric potential which does not adversely affect cells while causing the SAM to be reductively desorbed, and the electric potential can be appropriately determined according to the SAM to be used. The electric potential to be applied to the electrode substrate can be determined by measurement of cyclic voltammetry (CV). Specifically, an electric potential is applied to the electrode substrate having the SAM at a predetermined sweep rate from a predetermined starting electric potential to a predetermined switching electric potential. A turn is then made at the predetermined turning electric potential, and an electric potential is applied again at the predetermined sweep rate to the initial electric potential. Meanwhile, the current passing through the electrode substrate is measured, and a cyclic voltammogram is obtained on the basis of a relationship between the current and the applied electric potential. In the obtained cyclic voltammogram, an electric potential at which a peak of a negative current, i.e. a peak resulting from reductive desorption of the SAM is generated is determined. The electric potential to be applied to the electrode substrate in order to peel the SAM may be an electric potential at which the peak resulting from reductive desorption of the SAM is generated, or a more negative electric potential.

The electric potential to be applied to the electrode substrate may be a value selected from a range of −0.1 V to −2.0 V (vs Ag/AgCl), for example, while varying depending on the SAM to be used. For example, when the SAM comprises cysteine, an electric potential of −0.7 V or more, −0.8 V or more, −0.9 V or more, −1.0 V or more, −1.1 V or more, −1.2 V or more, −1.3 V or more, −1.4 V or more, −1.5 V or more, −1.6 V or more, −1.7 V or more, −1.8 V or more, or −1.9 V or more can be applied.

The time for application of an electric potential to the electrode substrate is not particularly limited as long as it is a time which is sufficient for the SAM to be peeled from the electrode substrate, and does not adversely affect cells, and for example, the time can be appropriately selected from a range of 5 to 100 seconds, preferably 30 to 60 seconds. Application of the electric potential to the electrode substrate can be performed continuously or intermittently.

Application of the electric potential to the electrode substrate can be performed in accordance with a conventional method. Specifically, a counter electrode, and a cell culture base material carrying cells after completion of cell culture are immersed in a culture medium or an appropriate buffer solution (e.g., PBS), and the counter electrode and the electrode substrate of the cell culture base material are connected to an appropriate power source, whereby the application of the electric potential to the electrode substrate can be performed.

By application of the electric potential to the electrode substrate, the SAM is peeled from the electrode substrate, whereby a cell-nanoscale thin film composite released from the electrode substrate can be obtained. To promote peeling of the SAM from the electrode substrate, and release of the cell-nanoscale thin film composite, operations of pipetting and shaking may be added.

3. Delivery of Cells Using Cell-Nanoscale Thin Film Composite

The cell-nanoscale thin film composite according to the present invention has excellent flexibility and self-supporting properties, and can be suctioned through a capillary having an inner diameter smaller than the diameter or the length of the longest diagonal of the cell-nanoscale thin film composite, and discharged from the capillary.

Examples of the “capillary” include glass needles, injection needles (syringe needles) and catheters. The size (gauge) and length of the “capillary” can be appropriately selected according to factors such as a size of the cell-nanoscale thin film composite, and a part in which the cell-nanoscale thin film composite is introduced.

Introduction of the cell-nanoscale thin film composite according to the present invention into the living body can be performed by suctioning the cell-nanoscale thin film composite together with a physiological saline solution from an injection needle or catheter tip, holding the cell-nanoscale thin film composite in an injection syringe or catheter, inserting the injection needle or catheter tip in an affected part or the vicinity thereof, discharging the cell-nanoscale thin film composite from the injection needle or catheter tip, and allowing the cell-nanoscale thin film composite to remain in place. By introduction of the cell-nanoscale thin film composite into the living body, carried cells can be delivered to the affected part or the vicinity thereof. One or more cell-nanoscale thin film composites can be introduced into the living body. In addition, the introduced patterned nanoscale thin film can be decomposed and absorbed in the living body.

For example, by introducing a cell-nanoscale thin film composite of retinal pigment epithelium (RPE) cells in the retina, a retinal lesion such as age-related macular degeneration can be treated. In an approach using a nanoscale thin film, a cell sheet can be stably disposed even on a complicated surface such as a retinal lesion part due to high flexibility of the nanoscale thin film. Further, in this approach, the recovered cell-nanoscale thin film composite can be subretinally transplanted in a minimally invasive manner with a syringe needle, and therefore an effect of preventing occurrence of a complication can also be expected.

4. Cell Culture Apparatus

The cell culture apparatus in the present invention may include a cell culture base material which is used for manufacturing the cell-nanoscale thin film composite; and a counter electrode and an appropriate power source which are used for applying an electric potential to an electrode substrate.

### EXAMPLE

The present invention will be described in further detail with an Example shown below, but the present invention is not limited to the Example.

1. Reagent

In this Example, the following reagents were used.
10-carboxydecanethiol (DOJINDO LABORATORIES)
7-carboxyheptanethiol (DOJINDO LABORATORIES)
L-cysteine (Wako Pure Chemical Industries, Ltd.)
polylactic acid-glycolic acid copolymer (75:25, PLGA, Polysciences, Inc.)
polyvinyl alcohol (molecular weight: 13,000 to 23,000, PVA, SIGMA-ALDRICH)
polydimethylsiloxane (PDMS, Dow Corning Toray Co., Ltd.)

All other reagents used were commercially available products.

2. Preparation of Cell Culture Base Material

2-1. Preparation of SAM Compound Solution

In this Example, experiments were conducted using three kinds of SAM compounds. SAM compound solutions were prepared under the following conditions.
10-Carboxydecanethiol was dissolved in ethanol to prepare a 1 mM solution.
7-Carboxyheptanethiol was dissolved in ethanol to prepare a 1 mM solution.
L-cysteine was dissolved in distilled water to prepare a 1 mM solution.
2-2. Preparation of Electrode Substrate

An electrode substrate was prepared in accordance with a method shown in FIG. 2.

Specifically, a silicone rubber sheet (thickness: 200 μm) was cut into a desired shape using a cutting plotter (Craft ROBO Pro, GRAPHTEC Corporation), and titanium was sputtered for 60 seconds with the silicone rubber sheet attached to a glass substrate. Gold was then sputtered for 60 seconds, and the silicone rubber sheet was then immersed at room temperature for 30 minutes in one of the SAM compound solutions prepared in “2-1. Preparation of SAM compound solution,” so that a SAM was formed on the gold surface. Finally, the silicone rubber sheet was peeled to obtain a gold-patterned electrode substrate having a SAM on a surface.

2-3. Electrochemical Evaluation of SAM

For the gold-patterned electrode substrate prepared in “2-2. Preparation of electrode substrate” and having a SAM comprising 10-carboxydecanethiol, 7-carboxyheptanethiol or L-cysteine, cyclic voltammetry (CV) measurement was performed using ALS Electrochemical Analyzer Model 760C (CH Instruments, Inc.).

For the measurement, a three-electrode type was used, where the electrode substrate, a reference electrode (Ag/AgCl electrode) and a counter electrode (Ag electrode) were each connected to ALS Electrochemical Analyzer, each of the electrodes was immersed in an aqueous KOH solution (0.5 M) subjected to nitrogen bubbling for 30 minutes, and CV measurement was performed under the following conditions.
scanning speed: 0.1 Vs⁻¹
segments: 2
sampling interval: 0.001 V
sensitivity: 1 e -4 AV⁻¹

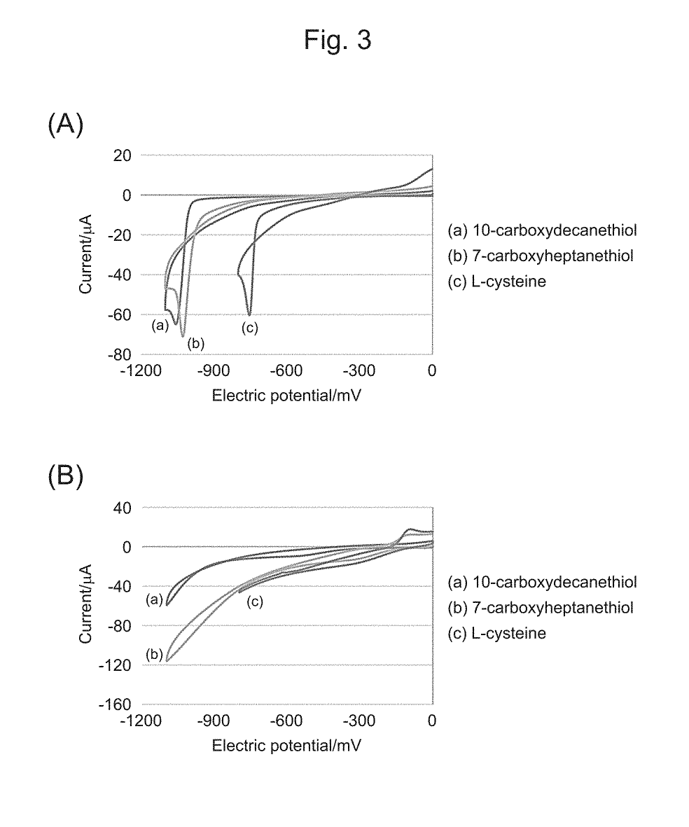

FIG. 3 shows cyclic voltammograms for electrode substrates having respective SAMs. In a first sweep (A), a peak was observed in each of graphs for electrode substrates having respective SAMs. On the other hand, in a second sweep (B), a peak was not observed in any of the graphs for electrode substrates. These results show that the SAM was peeled from each electrode substrate in the first sweep.

In addition, Table 1 below shows results of calculating from peak values in the cyclic voltammogram the number of SAM compound molecules peeled from the electrode substrate. The number of peeled SAM compound molecules was calculated on the basis of the total amount of charge at negative peaks in the cyclic voltammogram, and one electron being reacted per SAM compound molecule. Comparison with the theoretical value of the number of molecules originally bonded to the electrode substrate showed that 50 to 70% of molecules were peeled in all of three kinds of SAMs.

TABLE 1

| | | | Original | Number | Ratio (%) |
|---|---|---|---|---|---|
| | Density | Area | number of | of peeled | of peeled |
| SAM | (nmol/cm²) | (cm²) | molecules | molecules | molecules |
| 10-Carboxy- | 0.58 | 0.196 | 6.8E+13 | 3.3E+13 | 48.8 |
| decanethiol | | | | | |
| 7-Carboxy- | 0.45 | | 5.3E+13 | 3.8E+13 | 71.3 |
| heptanethiol | | | | | |
| L-cysteine | 0.38 | | 4.5E+13 | 3.2E+13 | 71.9 |

From these results, it was confirmed that it was possible to peel most of the SAM on the electrode substrate by applying a reduction electric potential.

From the results of the CV, it was confirmed that the SAM composed of L-cysteine was peeled from the electrode substrate at the lowest electric potential. This result showed that among three kinds of SAMs, the SAM composed of L-cysteine was most easily desorbed when an electric potential was applied. Subsequent experiments were conducted using L-cysteine for formation of a SAM.

2-4. Preparation of Cell Culture Base Material

A nanoscale thin film composed of PLGA was prepared in accordance with a method shown in FIG. 1.

(1) PVA was dissolved in distilled water at a concentration of 100 mg/mL to prepare a PVA solution. A glass substrate was spin-coated with the PVA solution at 4000 rpm for 40 seconds, and heated with a hot plate at 120° C. for 90 seconds.

(2) PLGA was dissolved in CH₂Cl₂ at a concentration of 20 mg/mL to prepare a PLGA solution. A stamp having a desired shape was prepared using PDMS, and the stamp was spin-coated with the PLGA solution at 4000 rpm for 40 seconds.

(3) The PLGA-coated stamp obtained in (2) was pressed against the PVA-coated glass substrate obtained in (1), and heating was performed for 90 seconds.

(4) The stamp was peeled from the substrate, and the glass substrate was immersed in water to dissolve PVA, so that a nanoscale thin film having a desired shape was released.

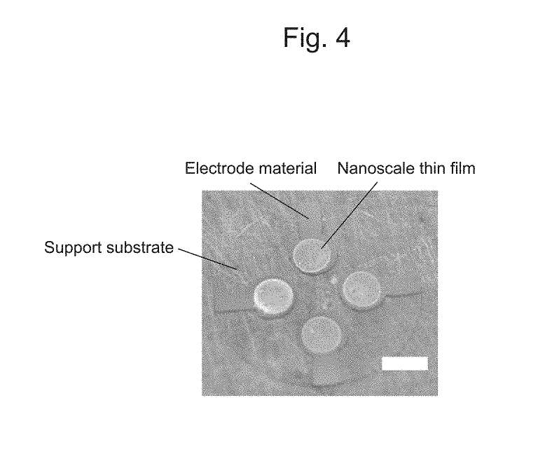

(5) On the electrode substrate prepared in “2-2. Preparation of electrode substrate” and having a SAM composed of L-cysteine on a surface, the released nanoscale thin film was placed using tweezers in water, the electrode substrate and the nanoscale thin film were taken out from water and dried, and the nanoscale thin film was adsorbed to obtain a cell culture base material in which a nanoscale thin film is provided on an electrode substrate with a SAM interposed therebetween (FIG. 4).

2-5. Peeling Test of Nanoscale Thin Film

The cell culture base material prepared in “2-4. Preparation of cell culture base material,” and a counter electrode (Pt electrode) connected to a −1.5 V dry battery were immersed in PBS, and an electric potential was applied to the electrode substrate for 30 to 50 seconds by the dry battery.

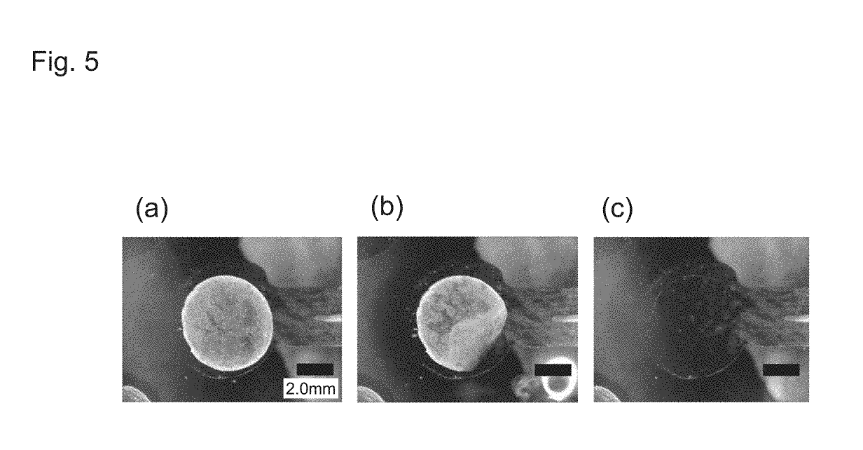

As a result, the area of part of the nanoscale thin film, which appeared black, increased (FIG. 5). This indicates that the SAM was peeled from the electrode substrate, and PBS entered between the nanoscale thin film and the electrode substrate. In this state, a water flow was lightly applied to the nanoscale thin film using a pipette, and resultantly, the nanoscale thin film was easily released from the electrode substrate.

On the other hand, when a water flow was applied while an electric potential was not applied, the nanoscale thin film was not peeled from the electrode substrate.

From these results, it was confirmed that it was possible to release the nanoscale thin film from the electrode substrate by utilizing reductive desorption of a SAM, and it was shown that the timing thereof was adjustable by manipulating application of an electric potential.

3. Peeling Test of Cell-Nanoscale Thin Film Composite

In this test, means for peeling the cell-nanoscale thin film composite from the substrate (SAM, PVA and temperature-responsive polymer) were compared and examined.

(1) Peeling Test Using SAM

In this test, the cell culture base material prepared in “2-4. Preparation of cell culture base material” was used. In cell culture, the cell culture base material was used after cell adhesiveness was improved by spin coating the nanoscale thin film with I-type collagen (5 mg/L) (at 4000 rpm for 40 seconds).

Retinal pigment epithelium cells (RPE-J cells) derived from a rat were prepared at a density of about 3×10⁶ cells/mL, and 400 μL of a suspension of the cells was added dropwise onto the nanoscale thin film of the cell culture base material. The cell culture base material was left standing in an incubator (Mini CO₂ Incubator Model 4020, Asahi Life Science Co., Ltd.) for about 1 hour until the cells were deposited on the nanoscale thin film, the culture medium was then removed, and PBS (−) was gently added to perform washing. PBS (−) was removed, a culture medium (500 mL of DMEM (Dulbecco's modified Eagle's Medium, High glucose, Wako Pure Chemical Industries, Ltd.)) with 5 mL of an antibiotic-antifungal agent (Antibiotic-Antimycotic, Gibco Corporation) and 20 mL of inactivated fetal bovine serum (FBS, BioWest) was added, and the cells were cultured in an incubator at 33° C.

After the cells were cultured for 2 days, an electric potential was applied to the electrode substrate for 30 to 50 seconds by a −1.5 V dry battery using the same method as described in “2-5. Peeling test of nanoscale thin film.”

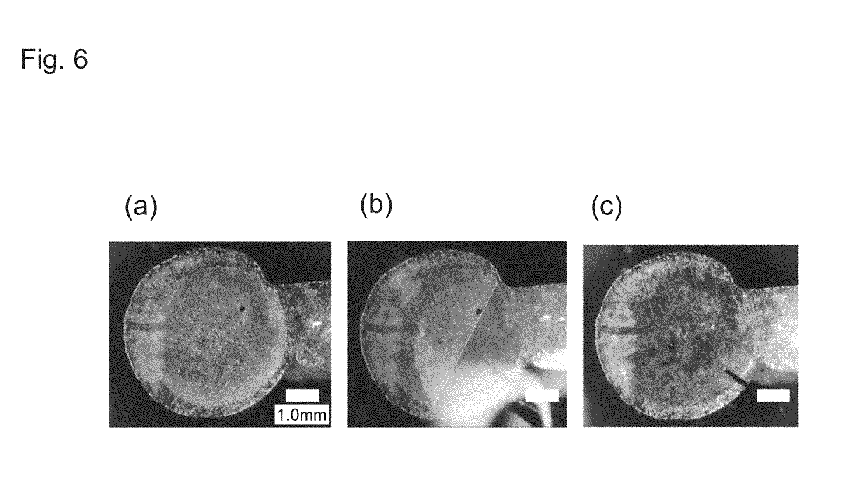

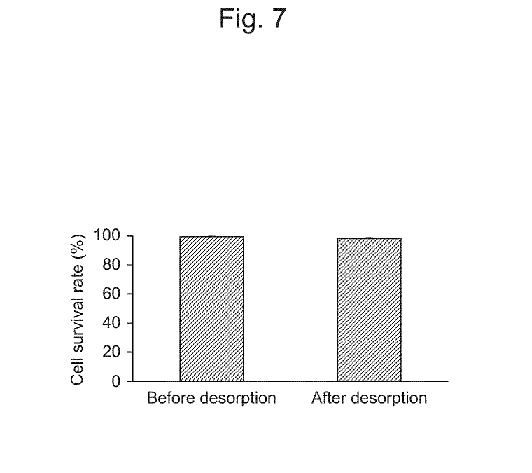

A water flow was lightly applied to the cell culture base material using a pipette, and resultantly, the cell-nanoscale thin film composite was easily released from the electrode substrate (FIG. 6: the area of part of the nanoscale thin film, which appeared black, increased (C) after application of the electric potential). In addition, survival of cells in the obtained cell-nanoscale thin film composite was confirmed from determination of life and death of cells using Calcein-AM and PI. Before and after desorption of the cell-nanoscale thin film composite from the electrode substrate, there was no change in tissue morphology, and the cell survival rate was approximately 100% (FIG. 7). These results showed that desorption operation utilizing reductive desorption of a SAM had almost no effect on cells on the nanoscale thin film.

(2) Peeling Test Using PVA of Sacrificial Layer

Experiments were conducted using a PVA layer as a substrate for the cell-nanoscale thin film composite during culture. An attempt was made to release the cell-nanoscale thin film composite from the substrate by gradual dissolution of the PVA layer in a culture solution.

Since it takes about 2 days until RPE-J cells become confluent on the nanoscale thin film, the substrate was prepared with the amount of PVA adjusted so as to fully dissolve the PVA layer in about 50 hours. Specifically, by the same method as described in “2-4. Preparation of cell culture base material,” PVA was dissolved in distilled water at a concentration of 10 mg/mL, 15 mg/mL, 20 mg/mL, 30 mg/mL, 50 mg/mL or 100 mg/mL to a prepare a PVA solution, and a glass substrate coated with PVA was prepared using the PVA solution. A stamp coated with PLGA was pressed against the glass substrate, heating was performed for 90 seconds, the stamp was peeled from the substrate, and the glass substrate was coated with I-type collagen to prepare a nanoscale thin film/PVA cell culture base material. Cells were seeded thereon, and cultured.

As a result, the nanoscale thin film was fully released to lose the substrate about 1 hour after the start of culture in the case of the nanoscale thin film/PVA cell culture base material prepared using a PVA solution at a concentration of 100 mg/mL, about 2 hours after the start of culture in the case of the nanoscale thin film/PVA cell culture base material prepared using a PVA solution at a concentration of 20 mg/mL, 30 mg/mL or 50 mg/mL, about 24 hours after the start of culture in the case of the nanoscale thin film/PVA cell culture base material prepared using a PVA solution at a concentration of 15 mg/mL, and about 36 hours after the start of culture in the case of the nanoscale thin film/PVA cell culture base material prepared using a PVA solution at a concentration of 10 mg/mL, and it was not possible to keep the nanoscale thin film adhering to the substrate during a period (about 2 days) until the cells became confluent.

Since the PVA dissolution rate, and the timing at which the nanoscale thin film is released may vary depending on factors such as the size of the nanoscale thin film, the concentration of a PVA solution to be used, and a culture period, it is difficult to release the nanoscale thin film from the substrate at a desired timing by this method using a PVA layer as the substrate.

(3) Peeling Test Using Temperature-Responsive Polymer

Experiments were conducted using a temperature-responsive polymer as a substrate for the cell-nanoscale thin film composite during culture. An attempt was made to release the cell-nanoscale thin film composite from the substrate by changing the adhesion property of the temperature-responsive polymer by changing the culture temperature.

The nanoscale thin film obtained in step (4) in “2-4. Preparation of cell culture base material” was placed on a commercially available culture plate Up Cell (Wako) with polyisopropyl acrylamide immobilized on a culture surface as a temperature-responsive polymer, the nanoscale thin film was coated with I-type collagen, and cells were then cultured thereon to prepare a cell-nanoscale thin film composite. Up Cell turns hydrophobic to form an adhesive surface in an environment at a temperature higher than 32° C., and turns hydrophilic to form a release surface in an environment at a temperature lower than 32° C.

After the cells were cultured for 2 days, Up Cell was left standing for several tens of minutes under an environment at a reduced temperature of 20° C., but it was not possible to release the cell-nanoscale thin film composite from Up Cell. In addition, a water flow was applied by pipetting, but only cells were peeled from Up Cell, and the cell-nanoscale thin film composite was not released. The cause of this may be that since the temperature of the cell culture environment was 33° C., and hence close to the boundary temperature (32° C.) in Up Cell, it was not possible to produce an appropriate temperature change; a strong intermolecular force acted between the nanoscale thin film composed of PLGA and Up Cell; and so on.

From the above results, it has been shown that the cell-nanoscale thin film composite can be easily released from the substrate at any timing by utilizing reductive desorption of a SAM. In other words, it has become evident that the method of the present invention which uses a SAM is remarkably useful for obtaining the cell-nanoscale thin film composite in comparison with other methods.

4. Use of Porous Electrode Substrate (Porous Film) as Electrode Substrate

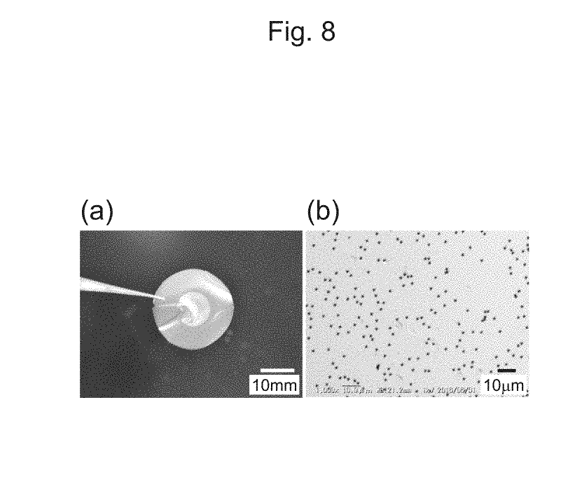

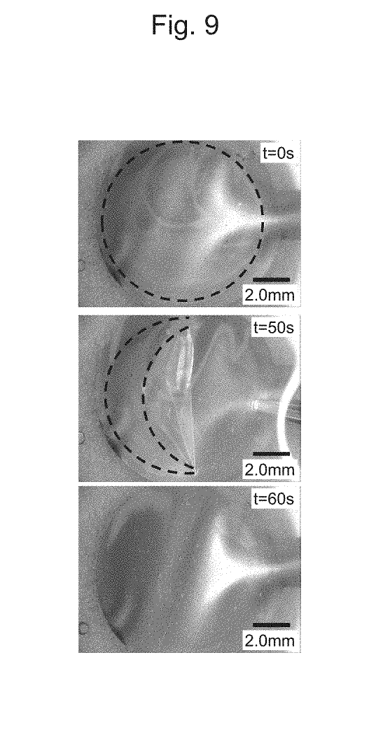

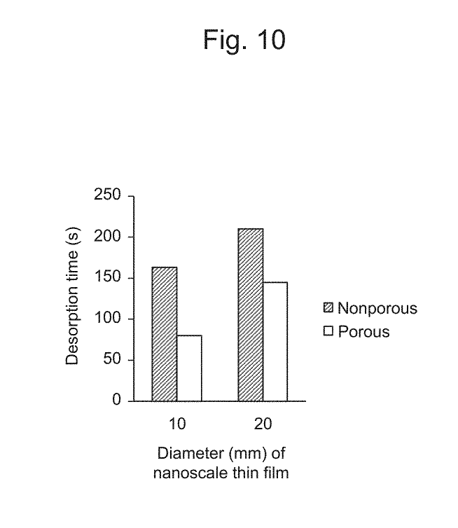

To accelerate the reductive desorption reaction of a SAM, it is necessary to quickly supply an electrolyte solution to an electrode surface. Thus, use of a porous film as an electrode substrate was considered. FIG. 8 illustrates a porous thin film (pore size: 1 μm) coated with gold in which FIG. 8a is a photograph and FIG. 8b is a SEM image. A SAM of L-cysteine was formed on the electrode surface, and a nanoscale thin film was then adsorbed onto the electrode. It was confirmed that when the reductive desorption reaction of the SAM proceeded, the nanoscale thin film was desorbed from the electrode substrate in about 1 minute (FIG. 9). FIGS. 9A, 9B and 9C illustrate states after 0 seconds, after 50 seconds and after 60 seconds, respectively. FIG. 10 illustrates a time required for desorption of the nanoscale thin film from the electrode substrate. The time required for desorption was evidently shorter for a porous electrode than for a nonporous electrode irrespective of whether the diameter of the nanoscale thin film is 10 mm or 20 mm.

This may be because in the porous electrode, the electrolyte solution is supplied not only from the edge of the electrode surface but also from the back side through pores, and therefore the reductive desorption reaction rapidly proceeds.

5. Delivery of Cell-Nanoscale Thin Film Composite Under Retina of the Eyeball in Rat

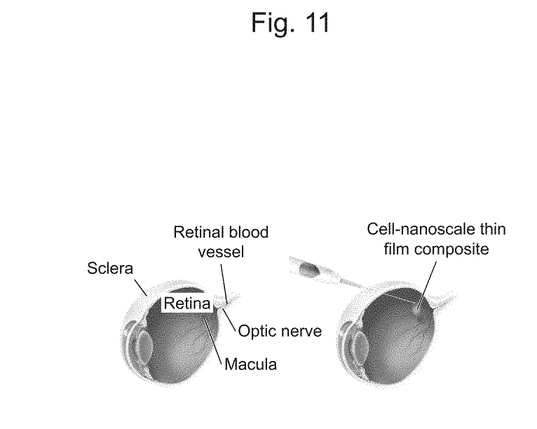

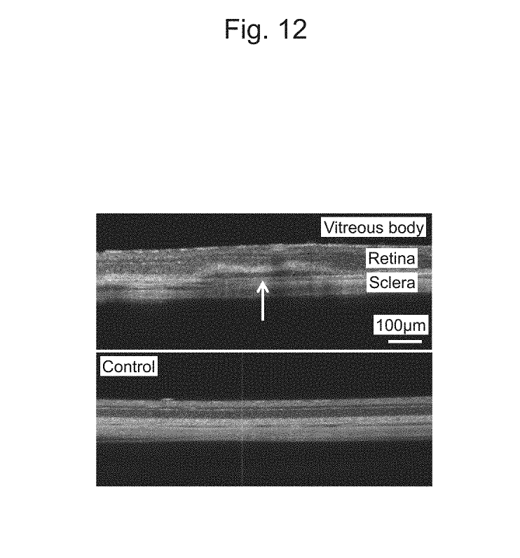

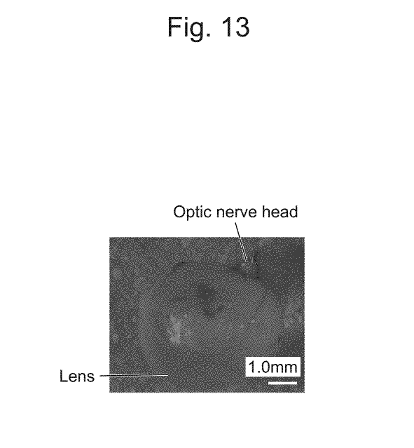

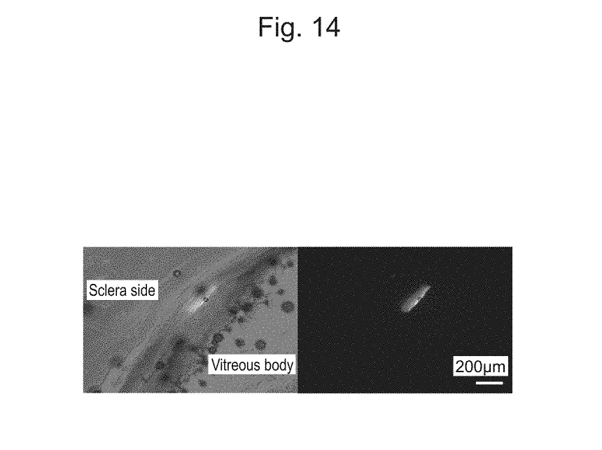

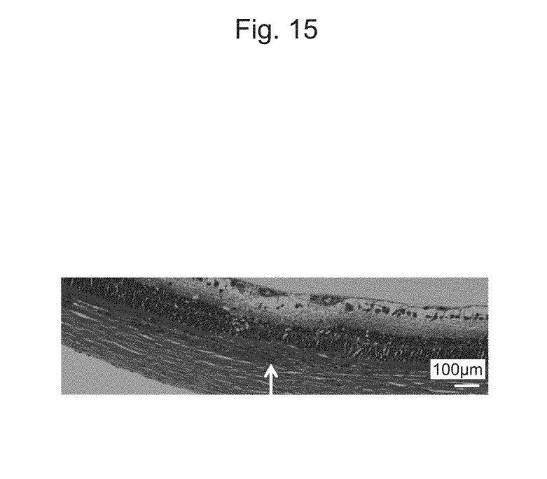

Delivery of the cell-nanoscale thin film composite desorbed from the electrode substrate under the retina of the eyeball in a rat was considered. After RPE cells (retinal pigment epithelium cells) formed a monolayer tissue on the nanoscale thin film, the monolayer tissue was desorbed as a cell-nanoscale thin film composite from the electrode substrate by reductive desorption of a SAM. Subsequently, the cell-nanoscale thin film composite was suctioned into a glass capillary needle, and the needle was then inserted to inject the cell-nanoscale thin film composite (FIG. 11). FIG. 12 illustrates an optical coherence tomography (OCT) image after the cell-nanoscale thin film composite is delivered subretinally. A shadow of a sheet-like structure was observed subretinally, but in the control retina, such a shadow was not observed. In addition, when the eyeball was extracted, a circular nanoscale thin film was observed in the posterior eye segment (FIG. 13). FIG. 14 is an image of a frozen section of the extracted eyeball, where it can be confirmed that the nanoscale thin film is delivered and spread subretinally. From histological examination performed using an image of another sample stained with hematoxylin eosin, it was indicated that cells were locally delivered subretinally by the nanoscale thin film (FIG. 15).

1. A method of manufacturing a cell-nanoscale thin film composite, the method comprising the steps of:
culturing a cell on a nanoscale thin film in a cell culture base material in which the nanoscale thin film is provided on an electrode substrate with a self-assembled monolayer interposed therebetween wherein the self-assembled monolayer is cysteine;
peeling the self-assembled monolayer from the electrode substrate by applying an electric potential to the electrode substrate so that the self-assembled monolayer is reductively desorbed from the electrode substrate; and
recovering a cell-nanoscale thin film composite released from the electrode substrate as the self-assembled monolayer is peeled.

2. The method according to claim 1, wherein the nanoscale thin film comprises a biocompatible polymer.

3. The method according to claim 2, wherein the biocompatible polymer is a polylactic acid-glycolic acid copolymer.

4. (canceled)

5. The method according to claim 1, wherein the electrode substrate is a porous material.

6. The method according to claim 5, wherein the porous material that is the electrode substrate is a porous film.

7. A cell culture base material in which a nanoscale thin film is provided on an electrode substrate with a self-assembled monolayer interposed therebetween wherein the self-assembled monolayer is cysteine.

8. The cell culture base material according to claim 7, wherein the electrode substrate is a porous material.

9. The cell culture base material according to claim 8, wherein the porous material that is the electrode substrate is a porous film.

10. A cell culture apparatus comprising a cell culture base material in which a nanoscale thin film is provided on an electrode substrate with a self-assembled monolayer interposed therebetween wherein the self-assembled monolayer is cysteine, and a counter electrode and a power source which are used for applying an electric potential to the electrode substrate.

11. The cell culture apparatus according to claim 10, wherein the electrode substrate is a porous material.

12. The cell culture apparatus according to claim 11, wherein the porous material that is the electrode substrate is a porous film.

13. A cell-nanoscale thin film composite in which a self-assembled monolayer which is cysteine, a nanoscale thin film and a cell are stacked in this order.
